SEQUENCE LISTING (8 sequences)

SEQ ID NO: 1 - DNA, 19 bp, synthetic construct
  source 1..19: mol_type = other DNA
agagctggtagcctgcctt

SEQ ID NO: 2 - DNA, 21 bp, synthetic construct
  source 1..21: mol_type = other DNA
ccggaagacgctttcttgagg

SEQ ID NO: 3 - DNA, 21 bp, synthetic construct
  source 1..21: mol_type = other DNA
ggagcgagatccctccaaaat

SEQ ID NO: 4 - DNA, 23 bp, synthetic construct
  source 1..23: mol_type = other DNA
ggctgttgtcatacttctcatgg

SEQ ID NO: 5 - DNA, 20 bp, synthetic construct
  source 1..20: mol_type = other DNA
ggggctcagcagtacgttag

SEQ ID NO: 6 - DNA, 19 bp, synthetic construct
  source 1..19: mol_type = other DNA
cggtggcgctgagcaattt

SEQ ID NO: 7 - DNA, 21 bp, synthetic construct
  source 1..21: mol_type = other DNA
tgggctacactgaggaccact

SEQ ID NO: 8 - DNA, 20 bp, synthetic construct
  source 1..20: mol_type = other DNA
gggagtgtctgttgaagtcg

(19) United States
(12) Patent Application Publication
(10) Pub. No.: US 2026/0335298 A1
(43) Pub. Date: Oct. 29, 2026

(54) PHARMACEUTICAL COMBINATION CONTAINING DONEPEZIL AND RIVASTIGMINE FOR PREVENTING, ALLEVIATING OR TREATING DEMENTIA OR COGNITIVE IMPAIRMENT, AND PREPARATION METHOD THEREFOR

(71) Applicant: G2GBIO, INC., Cheongju-si (KR)

(72) Inventors: Heeyong LEE, Daejeon (KR); Eunyoung SEOL, Daejeon (KR); Juhan LEE, Daejeon (KR); Hyejung JUNG, Seoul (KR); In Gyu KIM, Seoul (KR)

(21) Appl. No.: 19/114,892

(22) PCT Filed: Sep. 26, 2023
(86) PCT No.: PCT/KR2023/014929

§ 371 Date: Mar. 25, 2025

(30) Foreign Application Priority Data
Sep. 26, 2022 (KR) 10-2022-0121865

(51) Int. Cl. A61K 31/445; A61K 9/16; A61K 31/325; A61P 25/28

(52) U.S. Cl. CPC A61K 31/445; A61K 9/1647; A61K 31/325; A61P 25/28

(57) ABSTRACT

A pharmaceutical combination, composition or kit for preventing, improving, or treating dementia or cognitive disorder including donepezil or a pharmaceutically acceptable salt thereof and rivastigmine or a pharmaceutically acceptable salt thereof and having high drug compliance and excellent therapeutic effect and a preparing method thereof are provided.

### REFERENCE TO AN ELECTRONIC SEQUENCE LISTING

The contents of the electronic sequence listing (LPP20250,701US_SEQ.xml; Size: 7,951 bytes; and Date of Creation: Mar. 25, 2025) is herein incorporated by reference in its entirety. The contents of the electronic sequence listing in no way introduces new matter into the specification.

### TECHNICAL FIELD

The present invention relates to a pharmaceutical combination containing donepezil and rivastigmine for preventing, improving, or treating dementia or cognitive disorder, and a preparing method thereof.

### BACKGROUND

Due to an increase in an elderly population resulting from an extension of life expectancy, the number of dementia or cognitive disorder patients is rapidly increasing, and management of these patients is emerging as a serious social problem, which is increasing an economic burden on individuals and society.

In particular, more than 50% of senile dementia is a disease called Alzheimer's disease or Alzheimer type dementia (AD).

However, there is currently no therapeutic agent that may fundamentally cure the Alzheimer type dementia. However, as drugs that helps alleviate symptoms, donepezil (product name: Aricept) and rivastigmine (product name: Exelon) which are inhibitors of acetylcholinesterase that blocks decomposition of acetylcholine which is a neurotransmitter and memantine which is an N-methyl-D-aspartate (NMDA) receptor antagonist that prevents galantamine and glutamate from being excessively activated by binding to receptors are licensed and prescribed in many countries including the United States.

In addition, most elderly patients with dementia have difficulty swallowing drugs and have low drug compliance due to decreased cognitive ability, thereby leading to low fidelity to drugs. In fact, there is a research result showing that patients who took one drug pill have about 20% higher fidelity than those who took two drug pills have. As such, when a combination of therapeutic agent for dementia is developed, the fidelity to drug dosage and drugs may be increased.

According to previous studies, it was reported that when a combination of donepezil and memantine is prescribed for moderate to severe Alzheimer's dementia patients, all aspects of a cognitive function and behavioral scale scores were superior to a donepezil monotherapy group in a 24-week follow-up test result, and a combination therapy of the memantine and the donepezil has been approved by the FDA for treatment of moderate to severe Alzheimer's disease.

According to 2010 Dementia Disease Treatment Guidelines, it is recommended that the acetylcholinesterase inhibitor including the donepezil may be dosed in combination with the memantine which is an NMDA receptor antagonist, and that when efficacy of the donepezil is not observed or is insufficient in the monotherapy, another acetylcholinesterase inhibitor should be prescribed. In addition, the combination therapy of two or more types of the acetylcholinesterase inhibitors may increase side effects of the same series of drugs, and thus it is necessary to avoid the combination therapy.

Meanwhile, among the inhibitors of the acetylcholine esterase, the donepezil is prescribed as an oral preparation (tablet) dosed once a day or as a patch dosed once or twice a week, and the rivastigmine is prescribed as an oral preparation (capsule) dosed twice a day or as a patch dosed once a day. However, it is known that drug compliance is low due to gastrointestinal side effects, skin irritation side effects, and technical problems, and thus it is difficult to obtain sufficient therapeutic effects.

The once-daily oral preparation of donepezil is commercialized under a product name Aricept®, and a daily dosage is 5 mg, 10 mg, and 23 mg and is most commonly prescribed at 5 mg and 10 mg. The twice-daily oral preparation of rivastigmine is commercialized under a product name Exelon® and is prescribed at 3 mg, 6 mg, 9 mg, and 12 mg based on a daily dosage. Based on the daily dosage of the oral preparation, when the donepezil dosage is 23 mg, the rivastigmine dosage is 0.13 to 0.52 times, and when the donepezil dosage is 5 mg, the rivastigmine dosage is 0.6 to 2.4 times.

In order to solve the problems of the commercialized donepezil or rivastigmine products, a study to develop a combination therapeutic agent with an optimal weight ratio of the donepezil to the rivastigmine, which are the acetylcholinesterase inhibitors having high drug compliance and excellent therapeutic effect, has been proposed.

### SUMMARY

### Technical Problem

The present invention is directed to providing of a pharmaceutical combination for preventing, improving, or treating dementia or cognitive disorder having high drug compliance and excellent therapeutic effect when donepezil and rivastigmine are administered in combination, and a preparing method thereof.

### Technical Solution

As an aspect for achieving the above purpose, the present invention relates to a pharmaceutical combination for preventing, improving, or treating dementia or cognitive disorder including donepezil or a pharmaceutically acceptable salt thereof and rivastigmine or a pharmaceutically acceptable salt thereof.

As another aspect, the present invention relates to a pharmaceutical composition for preventing, improving, or treating dementia or cognitive disorder including an effective amount of donepezil or a pharmaceutically acceptable salt thereof and rivastigmine or a pharmaceutically acceptable salt thereof.

As still another aspect, the present invention relates to a kit including a pharmaceutical combination or a composition for preventing, improving, or treating dementia or cognitive disorder including an effective amount of donepezil or a pharmaceutically acceptable salt thereof and rivastigmine or a pharmaceutically acceptable salt thereof.

As a specific embodiment, the pharmaceutical combination, composition, or kit may include separate individual formulations including the donepezil or the pharmaceutically acceptable salt thereof and the rivastigmine or the pharmaceutically acceptable thereof in the effective amount, respectively, and each separate formulation may be administered simultaneously or sequentially. When formulated as the individual formulation, various administration routes and/or administration dosage forms may be selected. For example, it may be a combination of one or more formulations selected from a group consisting of an oral formulation, a patch formulation, and an injection formulation of the donepezil, and one or more formulations selected from a group consisting of an oral formulation, a patch formulation, and an injection formulation of the rivastigmine. Preferably, the separate individual formulation including the donepezil or the pharmaceutically acceptable salt thereof and the rivastigmine or the pharmaceutically acceptable salt thereof in the effective amount, respectively may be provided to an individual in the same dosage form and the same administration route.

As a specific embodiment, the pharmaceutical combination, composition or kit may include a single formulation including a mixture of the effective amount of the donepezil or the pharmaceutically acceptable salt thereof and the rivastigmine or the pharmaceutically acceptable salt thereof. As a specific embodiment, it may be prepared in a form of the same formulation including the donepezil or the pharmaceutically acceptable salt thereof and the rivastigmine or the pharmaceutically acceptable salt thereof of the pharmaceutical combination according to the present invention as a mixture, and it may be prepared in a form of separate individual formulations including each of the two active ingredients.

As a specific embodiment, the pharmaceutical combination according to the present invention may be formulated with the two active ingredients in tablet, patch or injection formulations, respectively.

As a specific embodiment, the pharmaceutical combination according to the present invention may be provided in a form of a mixture of the two active ingredients included in a microsphere, in a form of including a combination of the microspheres including each individual active ingredient, or in a form of formulation including a microsphere of one active ingredient, and the other active ingredient may be provided in a form of formulation different from that of the microsphere.

As a specific embodiment, a total content of the donepezil or the pharmaceutically acceptable salt thereof and the rivastigmine or the pharmaceutically acceptable salt thereof included in the pharmaceutical combination, composition or kit may be 0.1 to 99 wt. % based on a total weight of the pharmaceutical combination, composition or kit according to the present invention.

In addition, when the donepezil or the pharmaceutically acceptable salt thereof and the rivastigmine or the pharmaceutically acceptable salt thereof are included as the individual formulation in the pharmaceutical combination according to the present invention, they may be included in an amount of 0.1 to 65 wt. parts (based on donepezil) and in an amount of 0.1 to 50 wt. parts (based on rivastigmine), respectively, per 100 wt. parts of each individual formulation.

In addition, a weight ratio of the donepezil or the pharmaceutically acceptable salt thereof to the rivastigmine or the pharmaceutically acceptable salt thereof may be 1:0.003 to 1:1.3 based on a daily dosage as the donepezil and the rivastigmine, respectively. Preferably, the weight ratio of the donepezil or the pharmaceutically acceptable salt thereof to the rivastigmine or the pharmaceutically acceptable salt thereof may be 1:0.003 to 1:0.66, and more preferably, it may be 1:0.003 to 1:0.16.

As a specific embodiment, the pharmaceutical combination, composition or kit of the present invention may have an administration cycle at intervals of 1 day to 3 months, for example, may be administered once a day, once a week, once a month, once every two months, or once every three months.

As a specific embodiment, in the pharmaceutical combination for preventing, improving, or treating dementia or cognitive disorder including the donepezil or the pharmaceutically acceptable salt thereof and the rivastigmine or the pharmaceutically acceptable salt thereof of the present invention, AUC_{0-∞}/Cₘₐₓ of the donepezil and the rivastigmine is 5 hours or more when the donepezil and the rivastigmine are administered to an individual.

As still another aspect, the present invention relates to a method of preventing, improving, or treating dementia or cognitive disorder, including administering of the pharmaceutical combination, composition or kit including the effective amount of the donepezil or the pharmaceutically acceptable salt thereof and the rivastigmine or the pharmaceutically acceptable salt thereof.

### Advantageous Effects

A pharmaceutical combination according to the present invention can improve drug compliance by allowing combination dose of a formulation including donepezil or a pharmaceutically acceptable salt thereof and a formulation combining rivastigmine or a pharmaceutically acceptable salt thereof which continuously release drugs for one day or more to minimize side effects caused by rapid changes in blood drug concentration. In addition, therapeutic effect of dementia or cognitive disorder patients can be significantly improved due to the improvement of the drug compliance.

### BRIEF DESCRIPTION OF THE DRAWINGS

FIG. 1 is a graph showing a result of a pharmacokinetic test in which drug concentration was measured after administering microspheres of Examples 1 to 3 to rats. Examples 1 and 2 show a PK result of administration of a donepezil microsphere and a rivastigmine microsphere alone, and Example 3 shows a result of confirming a change in blood concentration after dosing a combined microsphere in which the donepezil microsphere and the rivastigmine microsphere are mixed.

FIG. 2 is a graph confirming a change in AChE mRNA expression level when treated with donepezil or rivastigmine alone in a SH-SY5Y cell line. In FIG. 2, when a p-value (significance probability) with respect to a control group corresponds to less than 0.001, it is indicated as ****.

FIGS. 3 and 4 are experimental results confirming effects of combined administration of donepezil and rivastigmine on AChE mRNA expression in a SH-SY5Y cell line according to Example 4. In FIG. 3, when a p-value (significance probability) with respect to a control group (group treated with donepezil alone) is less than 0.05, it is indicated as *, when the p-value is less than 0.005, it is indicated as ***, and when the p-value is less than 0.001, it is indicated as ****. In FIG. 4, when the p-value (significance probability) with respect to the control group (group treated with donepezil alone) is less than 0.01, it is indicated as **, when the p-value is less than 0.005, it is indicated as ***, and when the p-value is less than 0.001, it is indicated as ****.

FIG. 5 is an experimental result confirming effects of administration of rivastigmine alone on AChE mRNA expression in an APP-H4 cell line.

FIG. 6 is an experimental result confirming effects of combined administration of donepezil and rivastigmine on AChE mRNA expression in an APP-H4 cell line.

### BEST MODE

Hereinafter, the present invention will be described in detail.

Dementia is a neurodegenerative disease characterized by learning and cognitive deficit, and usually accompanied by behavioral, psychiatric, and motor symptoms. The dementia may be caused by a dementia-related disease such as Alzheimer's disease, Vascular dementia, Parkinson's disease, Lewy body dementia, Huntington's disease, Frontotemporal dementia, Creutzfeldt-Jacob disease, and Pick's disease and may be accompanied by the above diseases. Therefore, the dementia of the present invention may be Alzheimer's disease type dementia, Vascular dementia, Parkinson's disease type dementia, Lewy body dementia, Huntington's disease type dementia, Creutzfeldt-Jacob disease type dementia, or Pick's disease type dementia.

The cognitive disorder may include reduction in cognitive functions or cognitive areas, such as working memory, attention and vigilance, language learning and memory, visual learning and memory, reasoning and problem solving, such as executive function, processing speed, or social cognition. In particular, the cognitive deficit or cognitive disorder may refer to attention deficit, disorganized thinking, slow thinking, difficulty in understanding, poor concentration, impairment in problem solving, poor memory, difficulty in expressing thoughts and/or difficulty in integrating thoughts, feelings, and behaviors, or difficulty in extinction of irrelevant thoughts. The terms “cognitive deficit” and “cognitive disorder” may be used interchangeably to refer to a same thing. Examples of the cognitive disorder may include Alzheimer's disease, Schizophrenia, Parkinson's disease, Down syndrome, and Tourette's syndrome, but are not limited thereto.

The donepezil or a pharmaceutically acceptable salt thereof included in a pharmaceutical combination according to the present invention is known as a useful drug for preventing senile dementia, especially for preventing, improving, and treating the Alzheimer's disease. The donepezil or the pharmaceutically acceptable salt thereof is a drug approved for treatment of mild, moderate, and severe dementia. The donepezil is a reversible inhibitor of enzyme acetylcholinesterase and is also sold as hydrochloride under the trademark Aricept®.

Rivastigmine which is another active ingredient included in the pharmaceutical combination according to the present invention, is a drug approved for the treatment of mild, moderate and severe dementia of the Alzheimer's disease. The rivastigmine is a reversible cholinesterase inhibitor and is also sold as a tartrate salt under the trademark Exelon® and as a rivastigmine base under the trademark Exelon Patch®.

The pharmaceutically acceptable salt of the donepezil or the rivastigmine is not particularly limited thereto, but may preferably be an acid addition salt. The pharmaceutically acceptable salt should have low toxicity to a human body and should not adversely affect biological activity and physicochemical property of a parent compound. In addition, a free acid which may be used in preparation of the pharmaceutically acceptable salt may be divided into an inorganic acid and an organic acid. Hydrochloric acid, sulfuric acid, nitric acid, phosphoric acid, perchloric acid, and bromic acid may be used as the inorganic acid. Acetic acid, methanesulfonic acid, ethanesulfonic acid, p-toluenesulfonic acid, fumaric acid, maleic acid, malonic acid, pamoic acid, palmitic acid, stearic acid, phthalic acid, succinic acid, lactic acid, citric acid, citric acid, gluconic acid, tartaric acid, salicylic acid, malic acid, oxalic acid, benzoic acid, aspartic acid, and glutamic acid may be used as the organic acid. An organic base that may be used for preparing an organic base addition salt is tris(hydroxymethyl)methylamine and dicyclohexylamine. An amino acid that may be used for preparing an amino acid addition base is a natural amino acid such as alanine and glycine.

In addition, the donepezil or the rivastigmine according to the present invention may include not only the pharmaceutically acceptable salt but also all hydrates and solvates. The hydrates or the solvates may be crystallized or recrystallized by dissolving the donepezil or the rivastigmine in a solvent which may be mixed with water such as methanol, ethanol, acetone, or 1,4-dioxane, and then adding the free acid or the free base. In such a case, the solvates (especially hydrates) may be formed. Therefore, it is possible to include a stoichiometric solvate such as the hydrate in addition to various water-containing compounds that may be prepared by a method such as lyophilization as a compound of the present invention. The pharmaceutically acceptable salt of the donepezil may preferably be a hydrochloride salt, and the pharmaceutically acceptable salt of the rivastigmine may be a hydrochloride salt of the rivastigmine.

In a specific embodiment, the pharmaceutical combination may be for combined administration.

In a specific embodiment, the pharmaceutical combination may be a composition or a mixture including an effective amount of the donepezil or the pharmaceutically acceptable salt thereof and the rivastigmine or the pharmaceutically acceptable salt thereof, and the mixture may be in a form for administering two active ingredients together.

Alternatively, the pharmaceutical combination may be in a form in which the effective amount of the donepezil or the pharmaceutically acceptable salt thereof and the rivastigmine or the pharmaceutically acceptable salt thereof are included in separate individual formulations, respectively, and the individual formulations including each of the active ingredients may be administered simultaneously or sequentially.

In addition, the pharmaceutical combination may be in a kit form.

In a specific embodiment, a total content of the donepezil or the pharmaceutically acceptable salt thereof and the rivastigmine or the pharmaceutically acceptable salt thereof may be included in 0.1 to 99 wt. %, 1 to 90 wt. %, 1 to 85 wt. %, 1 to 75 wt. %, 1 to 50 wt. %, 5 to 85 wt. %, 10 to 80 wt. %, 20 to 70 wt. %, 30 to 60 wt. %, or 40 to 50 wt. % based on a total weight of the pharmaceutical combination according to the present invention, but is not limited thereto.

When the donepezil or the pharmaceutically acceptable salt thereof and the rivastigmine or the pharmaceutically acceptable salt thereof are included as the individual formulation in the pharmaceutical combination according to the present invention, the donepezil may be included in an amount of 0.1 to 65 wt. parts per 100 wt. parts of the individual formulation for the donepezil, and the rivastigmine may be included in an amount of 0.1 to 50 wt. parts per 100 wt. parts of the individual formulation for the rivastigmine.

Specifically, the pharmaceutical combination of the present invention may be administered orally or parenterally (e.g., intravenously, subcutaneously, intramuscularly, intraperitoneally, transdermally, nasally) through various administration routes depending on an intended method, and may be administered systemically or topically through such administration routes. In the present invention, when the pharmaceutical combination includes two separate individual formulations, the formulations including each active ingredient, for example, the donepezil or the pharmaceutically acceptable salt thereof, may be administered parenterally or orally via the same route or different routes and may preferably be administered orally. In addition, the rivastigmine or the pharmaceutically acceptable salt thereof may be administered parenterally.

In the pharmaceutical combination of the present invention, a suitable dosage of the donepezil or the pharmaceutically acceptable salt thereof and the rivastigmine or the pharmaceutically acceptable salt thereof varies depending on a patient's body weight, age, sex, health condition, diet, administration time, administration method, excretion rate, and severity of disease. As a specific example, a daily dosage of the donepezil of the present invention or the pharmaceutically acceptable salt thereof may be about 1 to 60 mg, and preferably 1 to 50 mg, 2 to 40 mg, 2 to 30 mg, 2 to 25 mg, 5 to 40 mg, 5 to 30 mg, 10 to 40 mg, 10 to 30 mg, or 25 to 50 mg based on the donepezil. In addition, a daily dosage of the rivastigmine of the present invention or the pharmaceutically acceptable salt thereof may be about 0.003 to 15 mg and preferably 0.015 to 3 mg based on the rivastigmine. However, the dosage of each ingredient may vary greatly depending on the conditions described above and is not limited by the examples described above.

In the pharmaceutical combination of the present invention, a suitable administration cycle of the donepezil or the pharmaceutically acceptable salt thereof and the rivastigmine or the pharmaceutically acceptable salt thereof may be determined according to the dosage. For example, the combination may be administered simultaneously, sequentially (in an arbitrary order), or in combination for preventing, improving, or treating dementia or cognitive disorder related diseases For example, the donepezil or the pharmaceutically acceptable salt thereof and the rivastigmine or the pharmaceutically acceptable salt thereof of the present invention may be administered once a day to once every 3 months, and preferably once a day, twice a week, once a week, once every 2 weeks, once a month, once every 2 months, or once every 3 months.

In a specific embodiment, a weight ratio of the pharmaceutical combination for preventing, improving, or treating dementia or cognitive disorder including the donepezil or the pharmaceutically acceptable salt and the rivastigmine or the pharmaceutically acceptable salt of the present invention based on the daily dosage may be 1:0.003 to 1:1.3, 1:0.003 to 1:1, 1:0.03 to 1:0.66, 1:0.003 to 1:0.5, 1:0.003 to 1:0.3, 1:0.003 to 1:0.16, 1:0.003 to 1:0.1, 1:0.003 to 1:0.05, 1:0.003 to 1:0.025, 1:0.003 to 1:0.02, 1:0.003 to 1:0.01, 1:0.01 to 1:0.025, 1:0.01 to 1:1, 1:0.01 to 1:0.66, 1:0.01 to 1:0.5, 1:0.01 to 1:0.3, 1:0.01 to 1:0.16, 1:0.01 to 1:0.1, 1:0.01 to 1:0.05, 1:0.01 to 1:0.025, 1:0.01 to 1:0.02, 1:0.01 to 1:0.015, 1:0.03 to 1:1, 1:0.03 to 1:0.66, 1:0.03 to 1:0.5, 1:0.03 to 1:0.3, 1:0.03 to 1:0.16, 1:0.03 to 1:0.1, 1:0.03 to 1:0.08, 1:0.03 to 1:0.05, 1:0.03 to 1:0.04, 1:0.05 to 1:1, 1:0.05 to 1:0.66, 1:0.05 to 1:0.5, 1:0.05 to 1:0.3, 1:0.05 to 1:0.2, 1:0.05 to 1:0.16, 1:0.05 to 1:0.15, 1:0.05 to 1:0.12, 1:0.05 to 1:0.1, 1:0.05 to 1:0.08, 1:0.05 to 1:0.075, 1:0.1 to 1:1, 1:0.1 to 1:0.66, 1:0.1 to 1:0.6, 1:0.1 to 1:0.5, 1:0.1 to 1:0.4, 1:0.1 to 1:0.3, 1:0.1 to 1:0.25, 1:0.1 to 1:0.2, 1:0.1 to 1:0.16, 1:0.1 to 1:0.15, 1:0.1 to 1:0.13, 1:0.1 to 1:0.12, 1:0.15 to 1:1, 1:0.15 to 1:0.66, 1:0.15 to 1:0.5, 1:0.15 to 1:0.4, 1:0.15 to 1:0.3, 1:0.15 to 1:0.25, 1:0.15 to 1:0.2, or 1:0.15 to 1:0.16.

For example, when the pharmaceutical combination is in the same oral dosage form or patch dosage form, the weight ratio based on the daily dosage of the donepezil and the rivastigmine may be 1:0.01 to 1:1, 1:0.01 to 1:0.66, 1:0.01 to 1:0.5, 1:0.01 to 1:0.3, 1:0.01 to 1:0.16, 1:0.01 to 0.1, 1:0.01 to 1:0.03, 1:0.02 to 1:0.025, 1:0.03 to 1:1, 1:0.03 to 1:0.66, 1:0.03 to 1:0.5, 1:0.03 to 1:0.3, 1:0.03 to 1:0.16, 1:0.05 to 1:1, 1:0.05 to 1:0.66, 1:0.05 to 1:0.5, 1:0.05 to 1:0.3, 1:0.05 to 1:0.16, 1:0.07 to 1:1, 1:0.07 to 1:0.66, 1:0.07 to 1:0.5, 1:0.07 to 1:0.3, 1:0.07 to 1:0.16, 1:0.1 to 1:1, 1:0.1 to 1:0.66, 1:0.1 to 1:0.5, 1:0.1 to 1:0.3, 1:0.1 to 1:0.16, 1:0.15 to 1:1, 1:0.15 to 1:0.66, 1:0.15 to 1:0.5, 1:0.15 to 1:0.3, or 1:0.15 to 1:0.2. The drug in the dosage form may be included in a free base form of the drug, or in a form of various salts, and in this case, a content of the drug included in the dosage form may be described based on the free base form of the drug.

In another embodiment, when the pharmaceutical combination is a same microsphere injection dosage form, it is preferable that a content of the rivastigmine is lower than that of the donepezil, for example, the weight ratio based on the daily dosage of the donepezil and the rivastigmine may be 1:0.003 to 1:0.66, 1:0.003 to 1:0.16, 1:0.003 to 1:0.13, 1:0.03 to 0.1, 1:0.03 to 1:0.03, 1:0.01 to 1:0.015, 1:0.02 to 1:0.3, 1:0.02 to 1:0.2, 1:0.02 to 1:0.1, 1:0.03 to 1:0.2, 1:0.03 to 1:0.2, 1:0.03 to 1:0.1, 1:0.04 to 1:0.3, 1:0.04 to 1:0.2, 1:0.04 to 1:0.1, 1:0.1 to 1:0.66, 1:0.1 to 1:0.2, 1:0.1 to 1:0.16, 1:0.15 to 1:0.3, 1:0.15 to 1:0.66, or 1:0.15 to 1:0.2. The drug in the microsphere may be included in the free base form of the drug or in the form of various salts, and in this case, the content of the drug included in the microsphere may be described based on the free base form of the drug.

The pharmaceutical combination according to the present invention has been confirmed to have a synergistic complementary effect of the donepezil or the pharmaceutically acceptable salt thereof and the rivastigmine or the pharmaceutically acceptable salt thereof which are acetylcholinesterase inhibitors, thereby exhibiting excellent efficacy in improving, preventing, or treating dementia and cognitive disorder related diseases. Therefore, the combination of the present invention for preventing, improving, or treating dementia or cognitive disorder related diseases may be efficiently used as a therapeutic strategy, and is particularly preferable for treating Alzheimer's disease.

In addition, the donepezil or the pharmaceutically acceptable salt thereof included in the pharmaceutical combination according to the present invention has efficacy of inhibiting accumulation of amyloid precursor protein. However, there is a problem that the efficacy of inhibiting the accumulation of the amyloid precursor protein also decreases due to an increase in a threshold due to long-term use, and the efficacy gradually decreases. In this case, by using the rivastigmine or the pharmaceutically acceptable salt thereof in combination with the donepezil or the pharmaceutically acceptable salt thereof, the decrease in the efficacy of the acetylcholinesterase inhibitor due to the increase in the threshold of the donepezil and the resulting decrease in the efficacy to inhibit the accumulation of the amyloid precursor protein may be complemented, thereby providing excellent improvement, prevention, or therapeutic efficacy of dementia and cognitive disorder related diseases. In particular, the pharmaceutical combination according to the present invention may exhibit the acetylcholinesterase inhibitory effect that is unpredictable when the donepezil and the rivastigmine are administered alone due to their combination, even though the rivastigmine is combined with the donepezil at an amount much lower than a normally used effective amount of rivastigmine for the treatment.

In particular, the pharmaceutical combination according to the present invention uses an amount similar to the effective amount of the normally used donepezil for the treatment. However, the pharmaceutical combination exhibits the acetylcholine esterase inhibitory effect that is unpredictable when the donepezil or the rivastigmine is administered alone due to combining the rivastigmine at an amount lower than the normally used effective amount of rivastigmine.

In the present invention, the donepezil or the pharmaceutically acceptable salt thereof and the rivastigmine or the pharmaceutically acceptable salt thereof included in the pharmaceutical combination of the present invention may be included in a separate individual formulation, respectively, or may be mixed and included in the same formulation. According to a method that may be easily performed by those having ordinary skill in the art to which the present invention pertains, the formulation is prepared by using a pharmaceutically acceptable carrier, thereby preparing in a form of a unit dose or preparing by being introduced in a multi-dose container. The pharmaceutically acceptable carrier is normally used in formulation and includes lactose, dextrose, sucrose, sorbitol, mannitol, starch, gum acacia, calcium phosphate, alginate, gelatin, calcium silicate, microcrystalline cellulose, polyvinylpyrrolidone, cellulose, water, syrup, methyl cellulose, methyl hydroxybenzoate, propyl hydroxybenzoate, talc, magnesium stearate, and mineral oil, but is not limited thereto. The pharmaceutical combination of the present invention may additionally include a lubricant, a wetting agent, a sweetening agent, a flavoring agent, an emulsifier, a suspending agent, a preservative, etc. in addition to the above ingredients.

In addition, the formulation may be a formulation for oral administration or parenteral administration having various forms known in the art. Examples of such formulation may include a powder, a granule, a tablet, a capsule, a suspension, an emulsion, a syrup, an aerosol, an external preparation, a suppository, a percutaneous absorption agent, or an injection preparation, but are not limited thereto. In the pharmaceutical combination according to the present invention, a composition or a mixture including the effective amount of the donepezil or the pharmaceutically acceptable salt thereof and the rivastigmine or the pharmaceutically acceptable salt thereof may be prepared in the same formulation form. Alternatively, in the pharmaceutical combination, the two active ingredients may be prepared in the separate individual formulations, and when prepared in the separate formulations, it may be prepared in a form of the same formulation or different formulations. Specifically, in an example of providing the different formulations including the two active ingredients as an individual ingredient, it may be provided in a combination of the oral formulation or a combination of the oral and parenteral formulations, for example a combination of the tablet formulation and the patch formulation.

As a specific embodiment, the formulation may be an injection formulation including a microsphere including drugs. In the microsphere, the active ingredients may be continuously released in a form in which the donepezil or the pharmaceutically acceptable salt thereof and/or the rivastigmine or the pharmaceutically acceptable salt thereof as the active ingredient may be evenly distributed or surrounded by a biodegradable polymer. The two active ingredients may be mixed to include in one microsphere, or each of individual microspheres including the individual active ingredient may be prepared, and it may be provided in a form of a combination of these microspheres, or one active ingredient may be provided in a form of formulation including the microsphere, and the other active ingredient may be provided in a form of formulation different from that of the microsphere.

In an embodiment of the present invention, when the individual microsphere formulation is prepared with the donepezil or the pharmaceutically acceptable salt thereof and/or the rivastigmine or the pharmaceutically acceptable salt thereof, the content of the drug in the microsphere, that is, the content of the donepezil may be 20 to 65 wt. % of the total microsphere, and the content of the rivastigmine may be 3 to 30 wt. % of the total microsphere. The drug in the microsphere may be included in the free base form of the drug or in the form of various salts, and in this case, the content of the drug included in the microsphere may be described based on the free base form of the drug.

The microsphere has an excellent effect in preventing or treating dementia or cognitive disorder related diseases due to high bioavailability despite a lower dosage compared to oral administration. The microsphere may have an average particle diameter of 10 μm to 150 μm.

Such a microsphere may be prepared using a method of preparing the microsphere well known in the art such as a solvent extraction and evaporation method, but is not limited thereto.

As a specific example, the pharmaceutical combination according to the present invention may include the microsphere including the donepezil or the pharmaceutically acceptable salt thereof and the microsphere including the rivastigmine or the pharmaceutically acceptable salt thereof, which are prepared separately. Alternatively, the microsphere including a mixture of the donepezil or the pharmaceutically acceptable salt thereof and the rivastigmine or the pharmaceutically acceptable salt thereof may be included in the pharmaceutical combination. Alternatively, in the pharmaceutical combination according to the present invention, a formulation in which only one of the donepezil or the pharmaceutically acceptable salt thereof and the rivastigmine or the pharmaceutically acceptable salt thereof is included in the microsphere and other active ingredients not included in the microsphere may be included in other forms of formulations (e.g., a percutaneous absorption agent).

The term “solvent extraction and evaporation method” used in the present invention refers to a method of preparing a biodegradable polymer microsphere by adding a biodegradable polymer solution prepared by dissolving the biodegradable polymer or the biodegradable polymer and the drug in the organic solvent to a continuous phase in a form of an aqueous solution including a surfactant to prepare an emulsion, extracting and evaporating the organic solvent from a dispersed phase in the emulsion in the continuous phase to form the microsphere, and then recovering the microsphere from the continuous phase.

A weight-average molecular weight of the biodegradable polymer is not particularly limited, but a lower limit may be 5,000 or more, preferably 10,000 or more, and an upper limit may be 500,000 or less, and preferably 200,000 or less.

A type of the biodegradable polymer is not particularly limited, but may be at least one selected from a group consisting of polyethylene glycol-poly(lactide-co-glycolide) block copolymer, polyethylene glycol-polylactide block copolymer, polyethylene glycol-polycaprolactone block copolymer, polylactide, polyglycolide, poly(lactide-co-glycolide), poly(lactide-co-glycolide) glucose, polycaprolactone and a mixture thereof, and more preferably, polylactide, poly(lactide-co-glycolide) and polycaprolactone may be used.

When the poly(lactide-co-glycolide) is used as the biodegradable polymer, a molar ratio of lactic acid to glycolic acid in the copolymer may be 99:1 to 50:50, and preferably 50:50, 75:25, or 85:15.

When the biocompatible polymer is included in two or more types, it may also be a combination or blend of polymers having different types of the polymers exemplified above, but it may also be a combination of polymers of the same type having different intrinsic viscosities, molecular weights, and/or monomer ratios (for example, a combination or blend of two or more poly(lactide-co-glycolide) having different intrinsic viscosities), or may be a polymer of the same type having different terminal groups (for example, a terminal group is an ester or a terminal group is an acid).

Examples of commercially available biodegradable polymers that may be used in the present invention include alone, combination, or blend of Evonik's Resomer series RG502H, RG503H, RG504H, RG502, RG503, RG504, RG653H, RG752H, RG752S, RG753H, RG753S, RG755S, RG756S, RG858S, R202H, R203H, R205H, R202S, R203S, R205S, and Corbion's PDL 02A, PDL 02, PDL 04, PDL 05, PDLG 7502A, PDLG 7502, PDLG 7504A, PDLG 7504, PDLG 7507, PDLG 5002A, PDLG 5002, PDLG 5004A, PDLG 5004, PDLG 5010, PL 10, PL 18, PL 24, PL 32, PL 38, PDL 20, PDL 45, PC 02, PC 04, PC 12, PC 17, PC 24, etc., but are not limited thereto. Suitable molecular weight or blending ratio of the biocompatible polymer applicable to the present invention may be appropriately selected by those skilled in the art in consideration of a decomposition rate of the biocompatible polymer and a drug release rate according thereto.

In a specific embodiment, in order to prepare the microsphere according to the present invention, the biocompatible polymer used in preparation of the dispersed phase is a PLA and PLGA polymer and may be one, or two or more selected from a group consisting of Purasorb PDL02A ((IV=0.16-0.24 dL/g; manufacturer: Corbion, Germany), Purasorb PDL04A ((IV=0.35-0.45 dL/g; manufacturer: Corbion, Germany), Purasorb PDLG7504A (IV=0.35-0.45 dL/g; manufacturer: Corbion, Germany), Resomer R205S (IV=0.55-0.75 dL/g; manufacturer: Evonik, Germany), Resomer R206S (IV=0.8-1.2 dL/g; manufacturer: Evonik, Germany), Resomer R203H (IV=0.25-0.35 dL/g; manufacturer: Evonik, Germany), Resomer RG 653H (IV=0.32-0.44 dL/g; Manufacturer: Evonik, Germany), Resomer RG 753H (IV=0.32-0.44 dL/g; Manufacturer: Evonik, Germany), Resomer RG755S (IV=0.50-0.70 dL/g; Manufacturer: Evonik, Germany), Resomer RG 756S (IV-0.71-1.0 dL/g; Manufacturer: Evonik, Germany), Resomer RG 858S (IV=1.3-1.7 dL/g; Manufacturer: Evonik, Germany), and Resomer RG 503H (IV=0.32-0.44 dL/g; Manufacturer: Evonik, Germany).

As a specific embodiment, the formulation may be a percutaneous absorption agent in a form of a reservoir or matrix including the donepezil or the pharmaceutically acceptable salt thereof and/or the rivastigmine or the pharmaceutically acceptable salt thereof as the active ingredient. Such percutaneous absorption agent has advantages of being safe, being relatively easy to store and handle during commercial production, being inexpensive, having excellent storage stability, being able to administer in an appropriate amounts of drugs for a long time, and being able to rapidly remove them from skin, and being able to avoid problems such as side effects that may occur in some cases relatively quickly. The percutaneous absorption agent may be prepared by a method well known in the art using various ingredients such as an adhesive polymer, an administration accelerator, and a plasticizer.

In the present invention, the term “individual” includes mammals, a human in particular, and an administration plan, administration interval, dosage, etc. may be easily set, changed, and adjusted by those skilled in the art based on the above-mentioned factors.

In the pharmaceutical combination of the present invention for preventing or treating dementia or cognitive disorder including the donepezil and the rivastigmine, each of the drugs may be continuously released for 1 day or more, 1 week or more, 2 weeks or more, 1 month or more, or 3 months or more.

As a specific embodiment, in the pharmaceutical combination of the present invention for preventing, improving, or treating dementia or cognitive disorder including the donepezil or the pharmaceutically acceptable salt thereof and the rivastigmine or the pharmaceutically acceptable salt thereof, AUC_{0-∞}/Cₘₐₓ of the donepezil and the rivastigmine may be 5 hours or more, respectively when the donepezil and the rivastigmine are administered to an individual.

In the present invention, AUC refers to an area under a plasma concentration-time curve which is a measure of total bioavailability, AUC_{0-∞} refers to an area under the plasma concentration-time curve up to infinite time, and Cₘₐₓ (maximum blood concentration) refers to a highest blood concentration of the administered drug when the drug is administered in vivo. In addition, all of these values are pharmacokinetic parameters for a single administration.

The pharmaceutical combination according to the present invention exhibits the AUC_{0-∞}/Cₘₐₓ value as described above, so that the blood concentration of the drug does not fluctuate excessively during administration of the drug and is maintained at a constant level while being therapeutically effective, and thus the pharmaceutical combination has an advantage of significantly reducing side effects commonly observed due to the acetylcholinesterase inhibitors such as the donepezil and the rivastigmine, for example, gastrointestinal side effects such as nausea, vomiting, abdominal pain, and duodenal ulcer, and side effects such as loss of appetite, confusion, insomnia, muscle cramps, weight loss, increased liver function level, bradycardia, heart block, or fainting.

As another specific embodiment, in the pharmaceutical combination of the present invention, the area under the blood concentration curve (AUC) of the donepezil may not be smaller than the AUC of the rivastigmine. Specifically, when the pharmaceutical combination according to the present invention is administered to an individual, the AUC of the rivastigmine is 100% or less, for example, 80%, 70%, 60% or 50% or less, based on the AUC value of the donepezil as 100%.

### EMBODIMENT OF INVENTION

Hereinafter, preferred examples of the present invention will be described in order to facilitate understanding of the present invention. It will be apparent to those skilled in the art, however, that the following examples are illustrative of the present invention and that various changes and modifications may be made within the scope and spirit of the present invention.

### Example 1: Preparation of a Donepezil Microsphere

A donepezil microsphere was prepared in order to confirm an effect of combined administration of donepezil and rivastigmine according to the present invention.

A dispersed phase was prepared by mixing 6.5 g of Resomer R203H (IV=0.32-0.44 dL/g; manufacturer: Evonik, Germany) which is a biocompatible polymer and 3.5 g of the donepezil with 17.11 g of dichloromethane (manufacturer: JTBaker, USA). The dispersed phase was sufficiently dissolved by stirring for 2 hours or more. A 0.5% (w/v) polyvinyl alcohol (viscosity: 4.8-5.8 mPa s) aqueous solution was used as a continuous phase, and 3,400 ml of the continuous phase was supplied to a membrane emulsifier while simultaneously injecting the prepared dispersed phase to prepare an emulsion, and then the emulsion was stirred at a speed of 200 rpm.

Temperature of the membrane emulsifier and the emulsion was maintained at 25° C., and when the injection of the dispersed phase was completed, the emulsion was stirred for 30 minutes, then heated to 45° C. and maintained for 3 hours, and an organic solvent was removed to produce the microsphere. After the removal of the organic solvent, the temperature of a microsphere suspension was lowered to 25° C. After repeatedly washing the microsphere suspension several times with distilled water, it was recovered and dried to prepare a biodegradable polymer microsphere including the donepezil.

### Example 2: Preparation of a Rivastigmine Microsphere

A dispersed phase was prepared by mixing 6.45 g of Resomer RG653H (IV=0.32-0.44 dL/g; manufacturer: Evonik, Germany) which is a biocompatible polymer and a pharmaceutically acceptable salt of the rivastigmine including 2.00 g of the rivastigmine with 32.29 g of dichloromethane (manufacturer: JTBaker, USA). The dispersed phase was sufficiently dissolved by stirring for 2 hours or more. A 1.0% (w/v) polyvinyl alcohol (viscosity: 4.8-5.8 mPa·s) aqueous solution was used as a continuous phase, and 4,836 ml of the continuous phase was supplied to a membrane emulsifier while simultaneously injecting the prepared dispersed phase to prepare an emulsion, and then the emulsion was stirred at a speed of 200 rpm.

Temperature of the membrane emulsifier and the emulsion was maintained at 25° C., and when the injection of the dispersed phase was completed, the emulsion was stirred for 30 minutes, then heated to 45° C., and maintained for 3 hours, and an organic solvent was removed to produce the microsphere. After the removal of the organic solvent, the temperature of a microsphere suspension was lowered to 25° C. After repeatedly washing the microsphere suspension several times with distilled water, it was recovered and dried to prepare a biodegradable polymer microsphere including the rivastigmine.

### Example 3: Preparation of a Combined Microsphere Including Donepezil and Rivastigmine

A combined microsphere was prepared by mixing the microspheres including donepezil and rivastigmine prepared in Example 1 and Example 2 in a weight ratio of 1.59:1.

### Experimental Example 1: Drug Content Test of a Donepezil Microsphere

To measure a content of the donepezil in the biodegradable polymer microsphere prepared in Example 1 and Example 3, 10 mg of the microsphere was completely dissolved in DMSO and then diluted with a mobile phase. 20 μl of the diluted solution was injected into HPLC and measured at a detection wavelength of 271 nm. A column used in the present experiment was Inertsil ODS-3, 5 μm, 4.6×150 mm, and the mobile phase was used by mixing phosphate buffer (pH 5.0) and acetonitrile in a ratio of 6:4 (v/v).

### Experimental Example 2: Drug Content Test of a Rivastigmine Microsphere

To measure a content of the rivastigmine in the biodegradable polymer microsphere prepared in Example 2 and Example 3, 10 mg of the microsphere was completely dissolved in DMSO and then diluted with a mobile phase. 20 μl of the diluted solution was injected into HPLC and measured at a detection wavelength of 210 nm. A column used in the present experiment was Inertsil ODS-3, 5 μm, 4.6×250 mm, and the mobile phase was used by mixing phosphate buffer (pH 6.0) and acetonitrile in a ratio of 65:35 (v/v).

The drug content in the biodegradable polymer microsphere tested in Experimental Example 1 and Experimental Example 2 is as shown in Table 1 below.

TABLE 1

Drug content (%, w/w)

| Category | Donepezil | Rivastigmine |
|---|---|---|
| Example 1 | 30.40 | — |
| Example 2 | — | 16.07 |
| Example 3 | 18.64 | 6.21 |

### Experimental Example 3: Pharmacokinetics (PK) Test of a Combined Microsphere Including Donepezil and Rivastigmine

To confirm a change in pharmacokinetics of the drug-containing microsphere, drug-containing microspheres prepared in Examples 1 to 3 were administered to rats, and then blood concentrations of the donepezil and the rivastigmine were measured. Conditions for administering the microspheres to rats are as shown in Table 2 below.

TABLE 2

| | Dosage of drug-containing | Dosage of drug |
|---|---|---|
| Category | microsphere (mg/head) | (mg/head) |
| Example 1 | 42.83 | 13.02 (donepezil) |
| Example 2 | 27.01 | 4.34 (rivastigmine) |
| Example 3 | 69.84 | 17.36 (donepezil 13.02, |
| | | rivastigmine 4.34) |

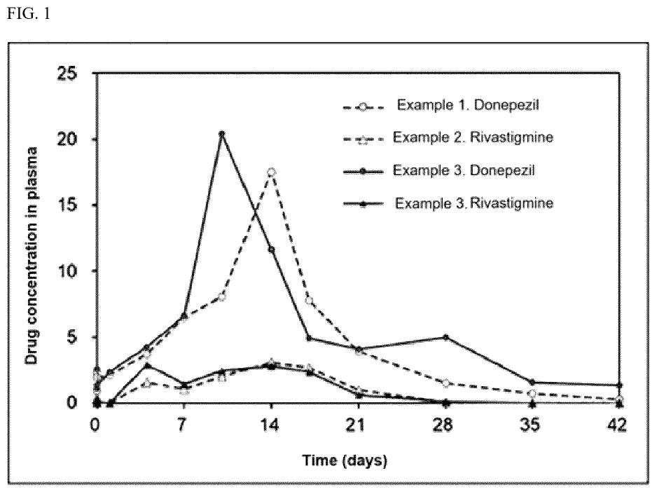

Results of measuring blood drug concentration after administering the microspheres including the drugs according to Examples 1 to 3 to rats according to the administration conditions of Table 2 are shown in FIG. 1. In FIG. 1, Example 1 and Example 2 are results of confirming a drug release pattern in experimental animals of the microspheres including the donepezil and the rivastigmine, respectively, and Example 3 is a result of confirming a release pattern of each drug in the experimental animals after administering the combined microsphere including the donepezil and the rivastigmine.

### Example 4: AChE mRNA Expression Pattern 1 According to Combined Administration of Donepezil and Rivastigmine

To establish cell's susceptibility to the donepezil and the rivastigmine and an optimal concentration range of each drug, AChE expression level analysis test according to treatment of each drug alone was conducted in a SH-SY5Y cell line which is a neuroblastoma that has been used for a long time as a research model for human neuron phenotype and Alzheimer's disease.

Specifically, the SH-SY5Y cell line was cultured in a 6-well plate (6×10⁵/well) using high-glucose Dulbecco's modified Eagle medium (DMEM) added with 10% fetal bovine serum and penicillin-streptomycin. SH-SY5Y cells were treated with the donepezil at final concentrations of 10, 20, 30, 40 (15.2 μg/ml) μM, or the rivastigmine at 0.01, 0.1, 0.5, 1, 5 (1.25 μg/ml) μM and cultured for 24 hours, and then, a total RNA was extracted from each cell using TRIzol, and cDNA was synthesized using a SuperScriptIII cDNA synthesis kit. Quantitative real-time PCR (qRT-PCR) was performed using the synthesized cDNA and primers and SYBR Green in Table 3 below using QuantStudio 3. In order to confirm amplification and accumulation of a target gene in real time, the qRT-PCR was performed under a condition that a cycle of 95° C. for 15 seconds and 60° C. for 1 minute was reacted 40 times after an initial denaturation reaction was performed at 50° C. for 2 minutes and 95° C. for 10 minutes.

TABLE 3

| | | Sequence |
|---|---|---|
| Name | Sequence | number |
| Human AchE | 5′-AGAGCTGGTAGCCTGCCTT-3′ | 1 |
| (forward) | | |
| Human AchE | 5′-CCGGAAGACGCTTTCTTGAGG-3′ | 2 |
| (reverse) | | |
| Human GAPDH | 5′-GGAGCGAGATCCCTCCAAAAT-3′ | 3 |
| (forward) | | |
| Human GAPDH | 5′-GGCTGTTGTCATACTTCTCATG | 4 |
| (reverse) | G-3′ | |

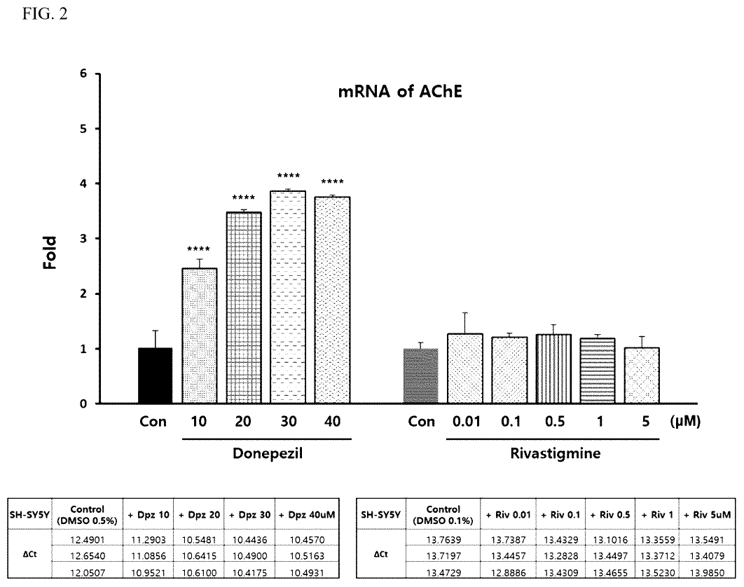

A gene expression level amplified by the Comparative Ct Method which is one of methods for quantifying a specific gene expression level was calculated, and analysis results are shown in FIGS. 2 to 4.

[Relational Expression of AChE mRNA Expression Level Analysis for Each Sample]

ΔCt value=Ct value of AChE in each sample−Ct value of reference gene (GAPDH) in the sample.

(Here, Ct refers to a cycle number in which amplification begins to increase clearly during a PCR process.)

As shown in FIG. 2, the AChE mRNA expression was dependently increased up to 20 μM donepezil concentration, but there was no difference beyond that, and no statistically significant change was observed by treatment with 0.01 to 5 μM rivastigmine. Through this, it was determined that the donepezil concentration of 20 μM or less, where AChE activity and quantitative increase according to administration of the donepezil found in clinical practice are clearly shown, was be suitable for a drug combination test.

Based on the experimental results, the donepezil and the rivastigmine were treated alone or in combination, and AChE mRNA expression level was analyzed in order to confirm effects of combined administration of the donepezil and the rivastigmine on AChE gene expression.

Specifically, the SH-SY5Y cell line was cultured in a 6-well plate (6×10⁵/well) using DMEM added with 10% fetal bovine serum and penicillin-streptomycin. Gene expression comparative analysis was performed by treating the SH-SY5Y cells with donepezil alone or with donepezil and rivastigmine in combination and performing qRT-PCR as in the previous experiment method.

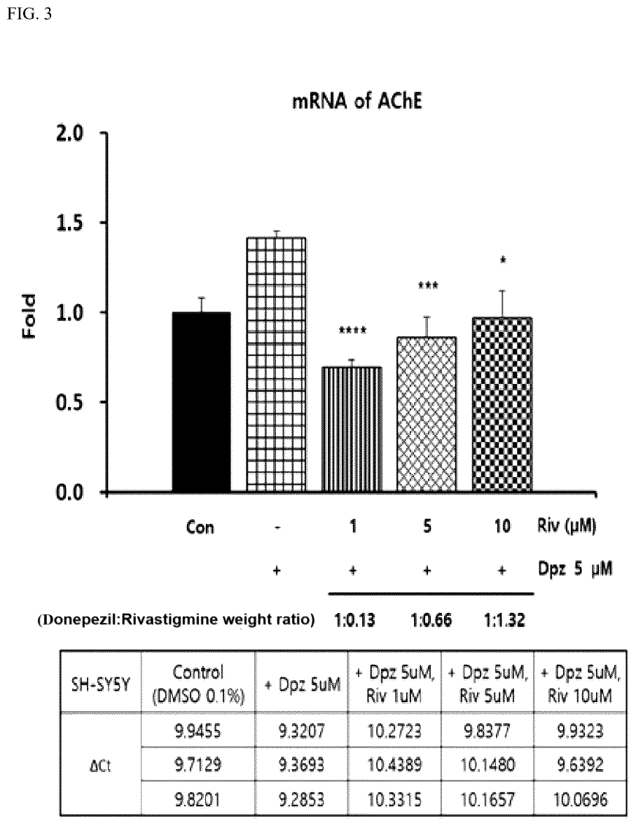

As shown in FIG. 3, the AChE mRNA expression increased by a treatment of 5 μM (1.9 μg/ml) donepezil alone was inhibited by combined treatment with 1, 5, and 10 (2.5 μg/ml) μM rivastigmine, and an inhibition effect of the AChE gene expression was highest when used in combination with 1 μM rivastigmine (weight ratio 1:0.13). However, as the rivastigmine drug concentration increased, the combination effect of the two drugs was nullified, showing low physiological/statistical significance in a donepezil:rivastigmine ratio of 1:1.32 (molar ratio 1:2).

According to the test results, the mRNA expression of AChE when donepezil 20 μM and low concentration rivastigmine were used in combination was comparatively analyzed. Specifically, an effect in a lower ratio range than a weight ratio of 1:0.13 which was confirmed as the most effective combination ratio in the FIG. 3 was confirmed.

Specifically, the gene expression comparative analysis was performed by treating the SH-SY5Y cells with donepezil 20 μM alone and with donepezil 20 μM and rivastigmine 0.1, 0.5, 1, and 5 μM as a final concentration in combination and performing qRT-PCR as in the previous experiment method.

As shown in the FIG. 4, when the donepezil and the rivastigmine were treated in combination at various concentrations, an AChE expression inhibition ability was confirmed in weight ratios of 1:0.003, 1:0.016, 1:0.03, and 1:0.16. In particular, the ratio of 1:0.016 specifically showed the greatest inhibitory effect, and it was confirmed that an optimal mixing ratio for preparing the pharmaceutical combination for preventing, improving, or treating dementia or cognitive disorder containing donepezil and rivastigmine was 1:0.016 based on the dosage.

### Example 5: AChE mRNA Expression Pattern 2 According to Combined Administration of Donepezil and Rivastigmine

An APP-H4 cell line is known as a dementia model cell line in which an abnormal segment of Amyloid Precursor Protein (APP) is increased by β-secretase and Alzheimer's disease and hyperphosphorylation of a Tau protein are induced by introducing a Swedish mutation of APP into human neuroglioblastoma H4 cells. In order to re-verify the effect of combined administration of the donepezil and the rivastigmine on the AChE mRNA expression and the drug combination ratio confirmed through a previous study, APP-H4 cells were treated with the donepezil and the rivastigmine alone or in combination as active ingredients, and the AChE gene expression was analyzed.

Specifically, the APP-H4 cell line was cultured in DMEM including 10% fetal bovine serum and gentamycine. Gene expression comparative analysis was performed by treating cells with donepezil 50 (19 μg/ml) μM alone or with donepezil 50 μM and rivastigmine 1 or 5 (1.25 μg/ml) μM as a final concentration in combination and performing qRT-PCR as in the previous experiment method (primers are shown in Table 4 below).

TABLE 4

| | | Sequence |
|---|---|---|
| Name | Sequence | number |
| Human AchE | 5′-GGGGCTCAGCAGTACGTTAG-3′ | 5 |
| (forward) | | |
| Human AchE | 5′-CGGTGGCGCTGAGCAATTT-3′ | 6 |
| (reverse) | | |
| Human GAPDH | 5′-TGGGCTACACTGAGGACCACT-3′ | 7 |
| (forward) | | |
| Human GAPDH | 5′-GGGAGTGTCTGTTGAAGTCG-3′ | 8 |
| (reverse) | | |

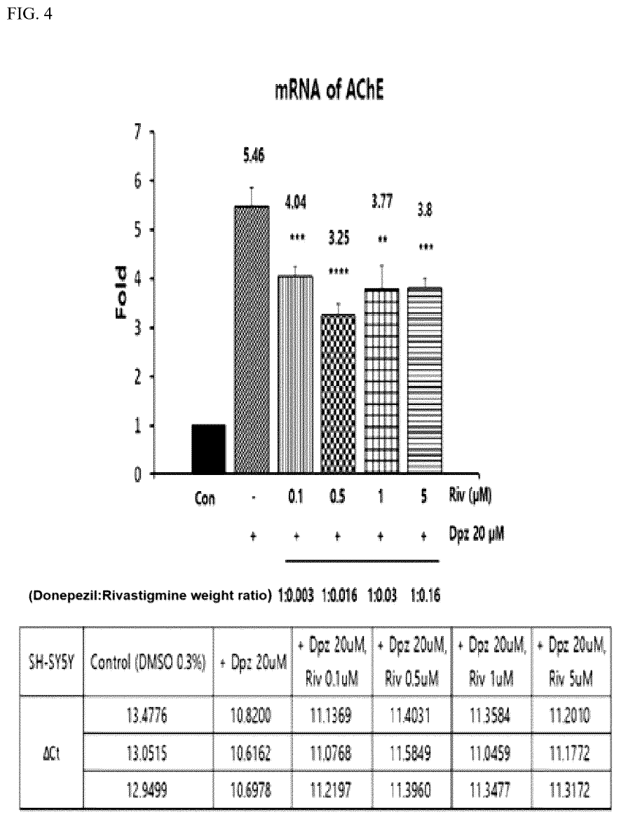

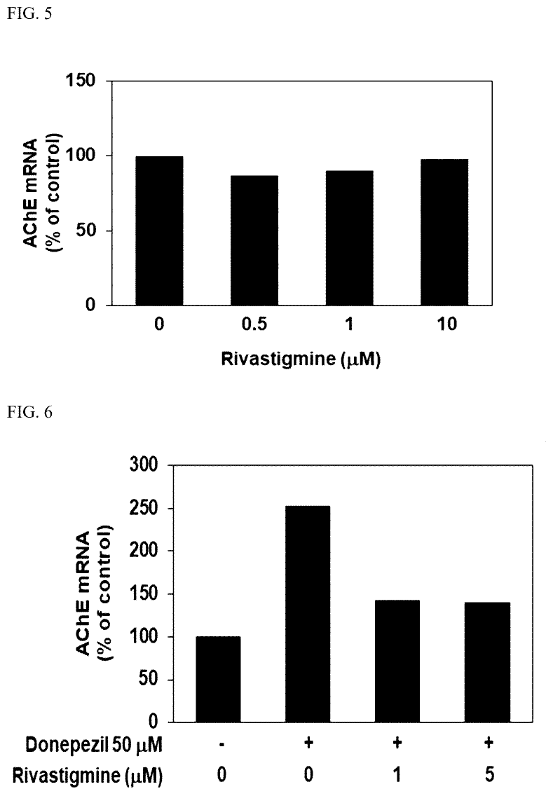

APP-H4 cells were treated with the donepezil and the rivastigmine alone or in combination and cultured for 24 hours, and then the AChE mRNA expression level was measured by qRT-PCR method. The results are shown in FIGS. 5 and 6.

As shown in FIG. 5, the rivastigmine was found to have no effect on the AChE mRNA expression in concentration range of 0.5, 1, or 10 μM.

As shown in FIG. 6, the AChE expression increased by 50 μM donepezil was reduced by combined treatment with 1 and 5 μM (1:0.13 and 1:0.66 based on weight ratio) rivastigmine, which was confirmed to be included in a range of donepezil:rivastigmine combination ratio confirmed through the previous experiment using SH-SY5Y.

In summary of the experiment results, it may be known that the AChE mRNA expression is not affected by inducing the AChE mRNA expression by the donepezil in human neuroblastoma cells and administration of the rivastigmine alone. However, when treating low-concentration rivastigmine that does not affect the AChE mRNA expression in combination with the donepezil, it was confirmed that there was a significant inhibitory effect on the AChE mRNA expression increased by the donepezil.

Both the donepezil and the rivastigmine are inhibitors of the acetylcholinesterase (AChE), with the donepezil exhibiting a mechanism of action of inhibiting acetylcholine degradation by the AChE alone, while the rivastigmine exhibits a mechanism of action of inhibiting acetylcholine degradation by the AChE and butyryl-cholinesterase (BuChE). Therefore, it should be understood that the donepezil and the rivastigmine theoretically do not affect an expression of a gene that produces the AChE itself, but it is true that there have been many reports in clinical research results on increases in AChE activity and quantity in patients taking the donepezil in a long-term. Regarding such a phenomenon, it is speculated that the decomposition of acetylcholine by AChE was inhibited by taking the donepezil, an amount of acetylcholine was maintained in the brain, and a regulatory mechanism for this increased the expression of the AChE gene for decomposing acetylcholine in vivo.

However, it was confirmed that when the rivastigmine is administered along with the donepezil in a certain ratio as in the present invention, the expression level of the AChE mRNA increased by the donepezil was effectively inhibited, thereby excellently inhibiting the expression of the AChE gene, which rather increases by the regulatory mechanism. In particular, in the case of the rivastigmine, it is determined that this effect is difficult to predict because it effectively inhibits the expression of the AChE mRNA when combined with the donepezil at the ratio according to the present invention even though the rivastigmine alone is administered in a content that does not affect the AChE. Moreover, the results of the present invention are the first to show that a weight-based combination ratio of the donepezil and the rivastigmine more effectively inhibits the expression level of the AChE mRNA at a ratio much lower than that of the commercialized dosage standard.

What is claimed is:

1. A pharmaceutical combination for preventing, improving, or treating dementia or cognitive disorder comprising donepezil or a pharmaceutically acceptable salt thereof and rivastigmine or a pharmaceutically acceptable salt thereof.

2. The pharmaceutical combination of claim 1, wherein the combination includes an individual formulation including the donepezil or the pharmaceutically acceptable salt thereof and an individual formulation including the rivastigmine or the pharmaceutically acceptable salt thereof, and the individual formulations are for simultaneously or sequentially administrated to a patient.

3. The pharmaceutical combination of claim 2, wherein the individual formulation including the donepezil or the pharmaceutically acceptable salt thereof and the individual formulation including the rivastigmine or the pharmaceutically acceptable thereof have the same dosage form or different dosage forms.

4. The pharmaceutical combination of claim 2, wherein each of the individual formulations in the pharmaceutical combination is an oral dosage form, a patch dosage form, or an injection dosage form.

5. The pharmaceutical combination of claim 1, wherein a weight ratio of a daily dosage of the donepezil to a daily dosage of the rivastigmine included in the pharmaceutical combination is 1:0.0031 to 1:1 as donepezil:rivastigmine.

6. The pharmaceutical combination of claim 4, wherein, when all individual formulations in the pharmaceutical combination are the oral dosage forms or the patch dosage forms, a weight ratio of a daily dosage of the donepezil to a daily dosage of the rivastigmine is 1:0.01 to 1:1 as donepezil:rivastigmine.

7. The pharmaceutical combination of claim 4, wherein, when all of the individual formulations of the pharmaceutical combination are the injection dosage forms, a weight ratio of a daily dosage of the donepezil to a daily dosage of the rivastigmine is 1:0.003 to 1:0.66 as donepezil:rivastigmine.

8. The pharmaceutical combination of claim 1, wherein a total content of the donepezil or the pharmaceutically acceptable salt thereof and the rivastigmine or the pharmaceutically acceptable salt thereof is 0.1 to 99 wt. % based on a total weight of the pharmaceutical combination.

9. The pharmaceutical combination of claim 1, wherein when the donepezil or the pharmaceutically acceptable salt thereof and the rivastigmine or the pharmaceutically acceptable salt thereof are each included as an individual formulation, the donepezil or the pharmaceutically acceptable salt thereof is included in an amount of 0.1 to 65 wt. parts as the donepezil, and the rivastigmine or the pharmaceutically acceptable salt thereof is included in an amount of 0.1 to 50 wt. parts as the rivastigmine based on 100 wt. parts of each of the individual formulations.

10. The pharmaceutical combination of claim 1, wherein a daily dosage of the donepezil or the pharmaceutically acceptable salt thereof is 1 to 60 mg as the donepezil, or a daily dosage of the rivastigmine or the pharmaceutically acceptable salt thereof is 0.003 to 15 mg as the rivastigmine.

11. The pharmaceutical combination of claim 1, wherein the rivastigmine compensates for a decrease in efficacy in which the donepezil or the pharmaceutically acceptable salt thereof inhibits accumulation of amyloid precursor protein.

12. The pharmaceutical combination of claim 1, wherein AUC_{0-∞}/Cₘₐₓ of the donepezil and the rivastigmine is 5 hours or more when the donepezil and the rivastigmine are administered to an individual.

13. The pharmaceutical combination of claim 1, wherein at least one of the donepezil or the pharmaceutically acceptable salt thereof and the rivastigmine or the pharmaceutically acceptable salt thereof is a formulation encapsulated in a microsphere.

14. The pharmaceutical combination of claim 13, wherein the pharmaceutical combination is a formulation in which the donepezil or the pharmaceutically acceptable salt thereof and the rivastigmine or the pharmaceutically acceptable salt thereof are encapsulated in the microsphere together, or
a mixed formulation including a formulation in which the donepezil or the pharmaceutically acceptable salt thereof is encapsulated in an individual microsphere and a formulation in which the rivastigmine or the pharmaceutically acceptable salt thereof is encapsulated in an individual microsphere.

15. A method for preventing, improving, or treating dementia or cognitive disorder comprising administering the pharmaceutical combination including the donepezil or the pharmaceutically acceptable salt thereof and the rivastigmine or the pharmaceutically acceptable salt thereof of claim 1, wherein a weight ratio of a daily dosage of the donepezil to a daily dosage of the rivastigmine included in the pharmaceutical combination is 1:0.0031 to 1:1 as donepezil:rivastigmine.
